(19) United States
(12) Patent Application Publication
(10) Pub. No.: US 2016/0067062 A1
(43) Pub. Date: Mar. 10, 2016

(54) PROSTHESIS COOLING LINER SYSTEM

(71) Applicants: THE JOHNS HOPKINS UNIVERSITY, Baltimore, MD (US); WALTER REED NATIONAL MILITARY MEDICAL CENTER, Bethesda, MD (US)

(72) Inventors: Matthew Jorgensen, Rockville, MD (US); Josh Charest, Lewiston, ME (US); Andrew Frazier, Mercer Island, WA (US); Michael Leddy, Princeton, NJ (US); Todd Sleeman, Silver Spring, MD (US)

(21) Appl. No.: 14/848,543

(22) Filed: Sep. 9, 2015

Related U.S. Application Data
(60) Provisional application No. 62/047,682, filed on Sep. 9, 2014.

(51) Int. Cl. A61F 2/78; A61F 2/80

(52) U.S. Cl. CPC A61F 2/7812; A61F 2/7843; A61F 2/80; A61F 2210/0057; A61F 2250/0003; A61F 2250/0013

(57) ABSTRACT

The present invention is directed to a prosthetic cooling liner system for reducing heat and sweat for an individual's residual limb. The prosthetic cooling liner system uses fluid conduit with a prosthetic silicone liner. The fluid conduit allows for cooling fluid to flow across the residual limb, cooling the skin's surface and thereby reducing sweat produced. The cooling fluid is contained in a reservoir in a small backpack worn by the individual. A peristaltic pump is used to pump the cooling fluid from the reservoir through the embedded tubes. Such a system allows the individual to maintain a high level of activity as well as comfort, while wearing the prosthetic.

### CROSS REFERENCE TO RELATED APPLICATIONS

This application claims the benefit of U.S. Provisional Patent Application No. 62/047,682 filed Sep. 9, 2014, which is incorporated by reference herein, in its entirety.

### FIELD OF THE INVENTION

The present invention relates generally to a medical device. More particularly, the present invention relates to a prosthesis cooling system.

### BACKGROUND OF THE INVENTION

Limb injuries that result in amputation are often treated with a prosthetic limb to provide mobility and function for the individual. Generally, the nature of the injury results in a loss of skin surface area, thereby decreasing the body's ability to cool itself. Many individuals remain highly active, continuing to run, hike, and participate in team sports. This high level of activity in combination with the nature of the injury leads to heat and sweat management issues. Sweat can lead to pistoning of the prosthesis and could possibly lead to the prosthesis falling off. Raised skin temperature can also have adverse effects on skin health under and around the prosthesis.

Accordingly, there is a need in the art for a prosthesis cooling system to reduce the skin temperature in and around the prosthesis.

### SUMMARY OF THE INVENTION

The foregoing needs are met, to a great extent, by the present invention which provides a system for cooling including a liner configured to be disposed between a residual limb of a user and a socket of a prosthetic limb. The system includes a conduit configured for flow of a cooling fluid. The conduit defines an inner lumen, and the inner lumen of the conduit accommodates the flow of the cooling liquid. The system also includes a pump in fluid communication with the conduit, such that the pump generates the flow of the cooling liquid through the conduit. A bladder in fluid communication with the conduit and the pump is also included. The bladder contains a supply of the cooling liquid.

In accordance with an aspect of the present invention, the liner is formed from one selected from a group of silicone, polyurethane, and a copolymer. The conduit takes the form of tubing. The tubing can be embedded in the liner. The tubing is formed from one selected from a group of silicone, polyurethane, and a copolymer. The conduit is arranged in a vertical alignment. The conduit terminates in a range of approximately 0.25 inches to 4 inches from a distal end of the liner. The pump takes the form of a peristaltic pump. A power source configured to provide power to the pump can be included in the system. The power source takes the form of a battery. The bladder includes inflow and outflow tubes, and wherein the inflow and outflow tubes are in fluid communication with the pump and the conduit. Alternately, the pump can take any suitable form known to or conceivable by one of skill in the art. It is also possible that the cooling fluid is moved or pumped through the system by the movement of the user, such as by walking, running, or other daily activities.

In accordance with another aspect of the present invention. The cooling fluid takes the form of one selected from a group of ice water, water, ice, gas, and liquid containing an additive. A backpack is included for housing the pump and bladder. The system can also include a charger for the battery. The pump can be actuated by one selected from a group consisting of walking, running, and movement. An on/off switch can be included and configured to provide control over power delivered to the system. The tubing can be positioned on an outside surface of the liner. The system can include a thermostat, wherein the thermostat is configured to cycle the system on and off in order to maintain the temperature of the system at a predetermined temperature. The liner can also define the lumen of the conduit.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram of a system for cooling a prosthetic limb, according to an embodiment of the present invention.

FIG. 2 illustrates an exploded view of a backpack component of a system for cooling a prosthetic limb, according to an embodiment of the present invention.

FIGS. 3A-3C illustrate front, back, and zoomed in views of the backpack component of the system for cooling a prosthetic limb according to an embodiment of the present invention.

FIG. 4 illustrates a back view of a user of the system, according to an embodiment of the present invention.

FIG. 5 illustrates a sectional view of a liner disposed around a residual limb within a prosthetic limb, according to an embodiment of the present invention.

FIGS. 6A-6N illustrate images of steps of manufacturing a prosthetic cooling liner, according to an embodiment of the present invention.

FIGS. 7A and 7B illustrate a sectional view of a setup for an experiment according to an embodiment of the present invention and a graphical view of results of the experiment, respectively.

FIG. 8 illustrates a graphical view of a comparison of the present invention with existing products.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains, having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a prosthetic cooling liner system for reducing heat and sweat for an individual's residual limb. The prosthetic cooling liner system uses embedded coolant tubes within a prosthetic silicone liner. The embedded tubes allow for cooling fluid to flow across the residual limb, cooling the skin's surface and thereby reducing sweat produced. The cooling fluid is contained in a reservoir in a small backpack worn by the individual. A peristaltic pump is used to pump the cooling fluid from the reservoir through the embedded tubes. Such a system allows the individual to maintain a high level of activity as well as comfort, while wearing the prosthetic.

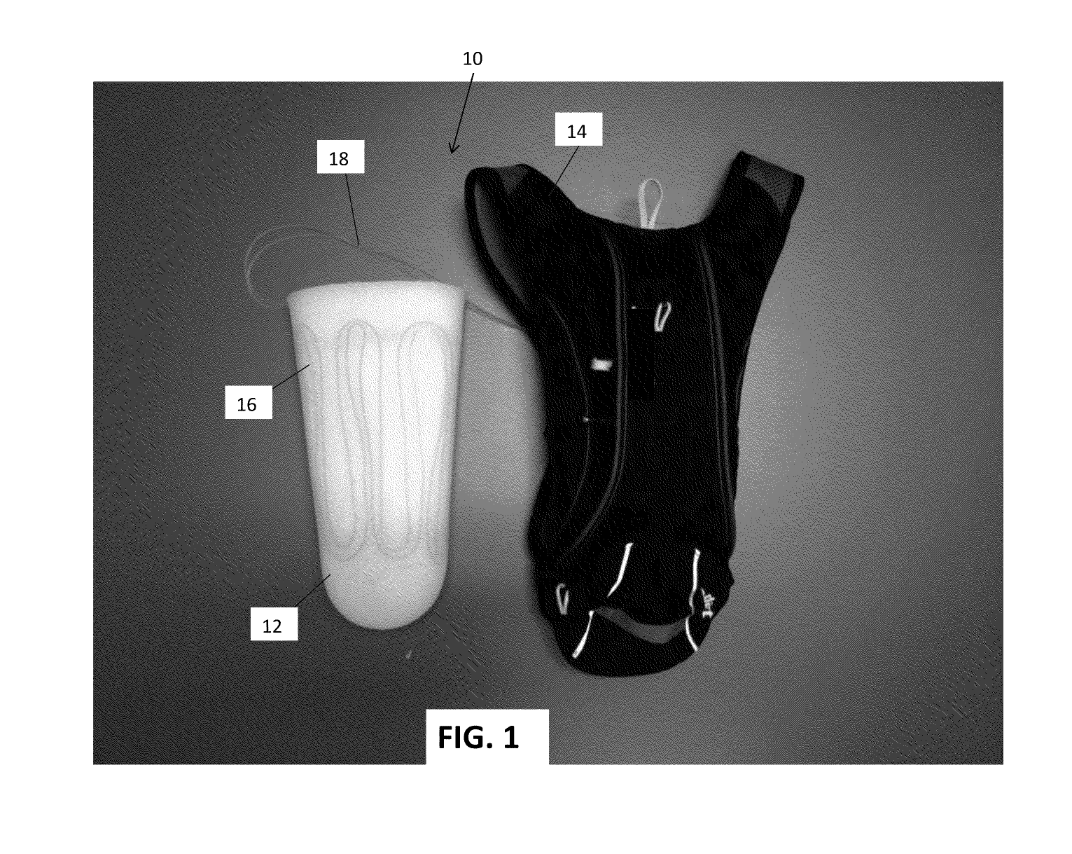

FIG. 1 illustrates a schematic diagram of a system for cooling a prosthetic limb, according to an embodiment of the present invention. As illustrated in FIG. 1, the system 10 includes a prosthetic cooling liner 12 and a backpack 14 configured for holding a reservoir of cooling liquid and a pump for generating a flow of the cooling liquid through the system. The prosthetic cooling liner 12 includes fluid conduit 16 through which the cooling liquid flows. The prosthetic cooling liner 12 and the fluid conduit 16 can both be formed from silicone, polyurethane, a copolymer, or any other suitable material, known to one of skill in the art. The fluid conduit 16 can take the form of separate embedded tubing, a lumen for fluid movement defined by the liner itself, or any other suitable form known to or conceivable by one of skill in the art. The fluid conduit 16 defines a fluid flow path through which the cooling fluid travels through the system. The size of the liner can be dictated by the type of residual limb and the size of the user. Each liner could be custom made for each individual user or general shapes and sizes could be established for each type of residual limb.

Further as illustrated in FIG. 1, connector tubing 18 connects the liner 12 to the backpack 14 to allow for the flow of cooling liquid generated by the pump, such that the fluid conduit 16 and the connector tubing are in fluid communication with each other, the reservoir, and the pump. While a backpack is shown as an example herein, the reservoir and the pump can be disposed in any other suitable housing known to or conceivable by one of skill in the art. The housing should, however, in preferred embodiments be portable and lightweight, in order to ensure that it can be carried with the user during exercise, strenuous activity, or daily tasks.

As illustrated in FIG. 1, the fluid conduit preferably has a vertical arrangement in some embodiments, with the fluid conduit extending from a proximal end of the liner 12 to a distal end of the liner 12 and then curving back in the other direction. The fluid conduit is aligned vertically to mitigate the inner diameter of the fluid conduits from compressing due to radial expansion. Preferably, the fluid conduit is not positioned over or immediately surrounding the bone of the residual limb, for instance, on or immediately surrounding the tibia. This arrangement is further facilitated by the vertical alignment of the fluid conduit. The fluid conduit also preferably stops a predetermined distance from the distal end of the liner to prevent the user's weight from compressing the fluid conduit during movement. In a preferred embodiment the conduit terminates approximately 2 inches from a distal end of the liner. Alternately, the conduit can terminate in a range of approximately 0.25 inches to approximately 4 inches from a distal end of the liner. The fluid conduit exits the liner at the same point at the top of the liner and is connected to the backpack component with barbed fitting. Any suitable cooling fluid can be used. It is also possible that cooled air or other cooling gas could be used as the cooling fluid. It is possible that in the closed loop system of the present invention, the cooling fluid can be run through the fluid conduit an infinite number of times, while the user participates in an activity, exercise, or daily tasks, without having to add to or replace the fluid. It is therefore, preferable, that the cooling fluid be able to maintain cooling properties for a prolonged period of time. If cooling fluids other than water are used, it is possible that these cooling fluids be provided in a packet, bottle, compressed gas canister, or other suitable means of storage and delivery. In another possible embodiment, an additive packet can also be provided in order to improve or prolong the cooling properties of the cooling fluid. The fluid can also be run past a device for cooling, in order to maintain the temperature of the fluid, especially during extended use. In another possible embodiment, cleaning agents could also be provided for running through the system, in order to promote optimal function of the system. However, preferably, easily accessible materials such as water and/or ice are used to fill the reservoir for cooling.

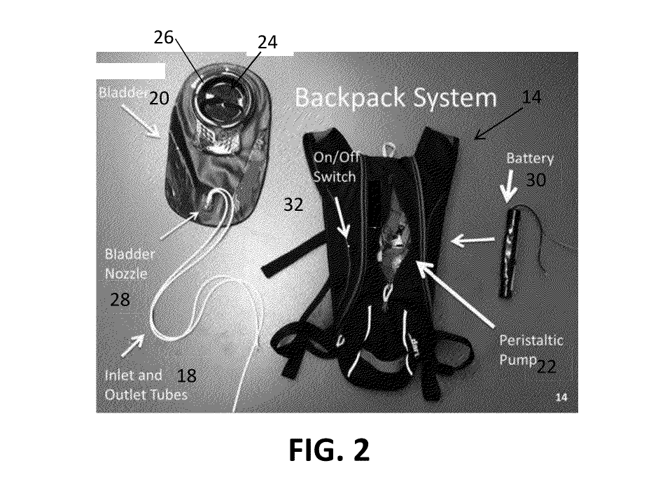

FIG. 2 illustrates an exploded view of a backpack component of a system for cooling a prosthetic limb, according to an embodiment of the present invention. As illustrated in FIG. 2, the backpack component 14 includes connector fluid conduit 18 that is configured to connect the reservoir 20, the pump 22, and the fluid conduit (not shown). The reservoir 20 includes an opening 24 for receiving the cooling liquid that is preferably sealable with a water tight closure 26, such that the system is portable without leakage of cooling fluid. The reservoir 20 also includes a nozzle 28 through which the cooling fluid can exit the reservoir 20. The nozzle 28 is coupled to the connector tubing 18. One connector tube runs to the pump 22 and one runs to the liner and returns the cooling fluid from the liner after it has circulated around the limb. The pump 22 is also connected to the liner with a tube. Preferably, the pump 22 takes the form of a peristaltic pump. The pump 22 and the reservoir 20 are in fluid communication. The backpack component 14 also includes a battery 30 for powering the pump 22. The battery 30 can take the form of a replaceable or rechargeable battery, or any other suitable, portable power source known to or conceivable by one of skill in the art. A port can be included on the backpack to facilitate charging. The backpack component 14 also includes an on/off switch 32 that can be used to power on the system when needed and turn it off when it is not needed. To load the backpack component 14 with the cooling fluid, such as ice water, the user simply removes the reservoir 20, places ice cubes and water into the reservoir 20 and inserts it back into the backpack 14. All of the tube connections are held together with plastic barbed fittings, or other suitable means of connection known to or conceivable by one of skill in the art. While a backpack is shown as an example herein, the backpack component can take the form of any other suitable means to provide portability to the system. For instance, a waist pack could be used or a device that secures the system to the prosthetic limb of the user.

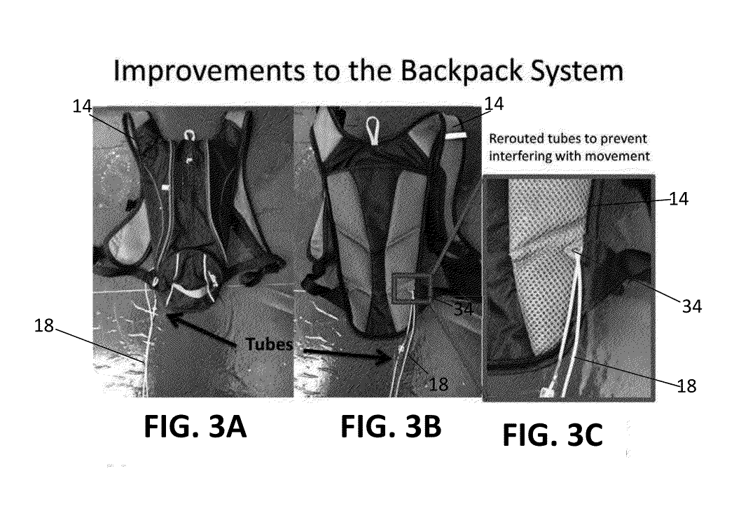

FIGS. 3A-3C illustrate front, back, and zoomed in views of the backpack component of the system for cooling a prosthetic limb according to an embodiment of the present invention. The backpack component 14 houses in part the connector tubing 18. The connector tubing 18 exits the backpack through a small hole 34 defined by the material of the backpack 14. Preferably, the hole is reinforced. The hole 34 is positioned at the back of the backpack 14 in order to prevent the connector tubing 18 from interfering with the movement of the user. The connector tubing 18 can also be arranged in any other suitable fashion known to or conceivable by one of skill in the art to prevent interference with motion and also to prevent kinking of the tubing.

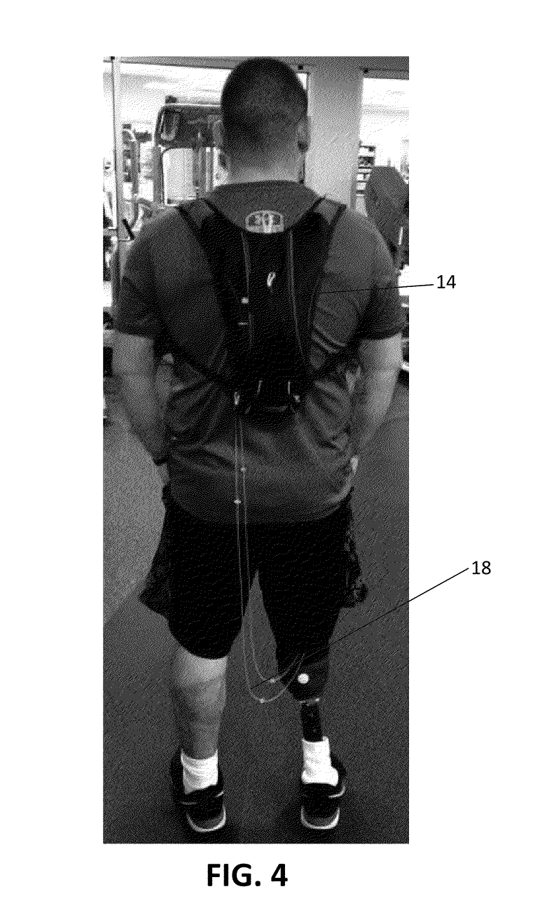

FIG. 4 illustrates a back view of a user of the system, according to an embodiment of the present invention. As shown in FIG. 4, the user wears the backpack 14 on his back. The liner component (not shown) is disposed within the user's prosthetic limb and surrounding the user's residual limb, or a secondary liner chosen by the user. The connector tubing 18 connects the backpack component 14 to the liner in such a way that it will not impede the user's movement. The system can also include a thermostat for monitoring the temperature of the system and/or the user's residual limb. The thermostat can be configured to cycle the system on and off to maintain the system and/or the user's residual limb at a preferred temperature, predetermined and set on the thermostat by the user.

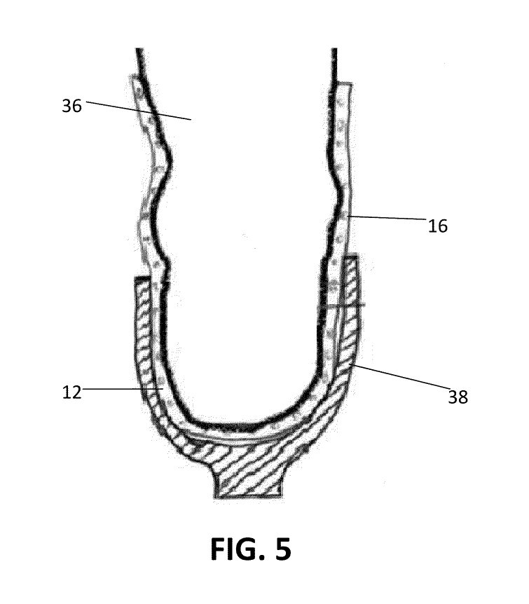

FIG. 5 illustrates a sectional view of a liner disposed around a residual limb within a prosthetic limb. As illustrated in FIG. 5, the liner 12 surrounds the residual limb 36. The liner 12 is disposed between the residual limb 36 and the prosthetic 38. Fluid conduit 16 is disposed within the wall of the liner 12 to allow for cooling fluid to flow around the residual limb 36.

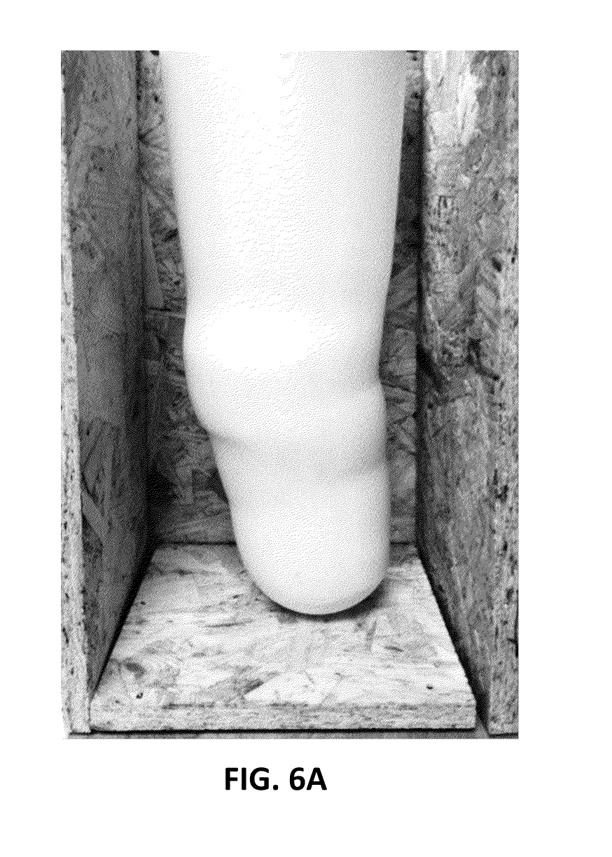

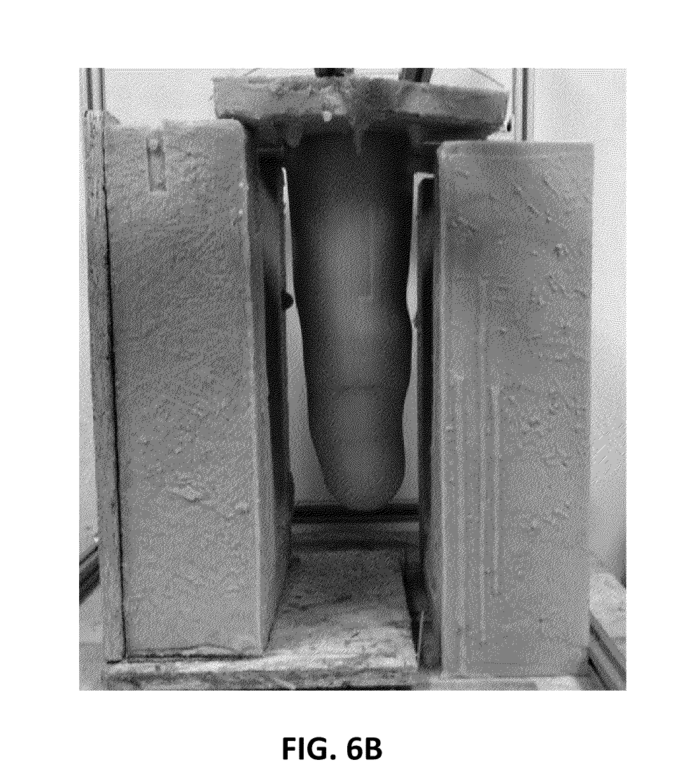

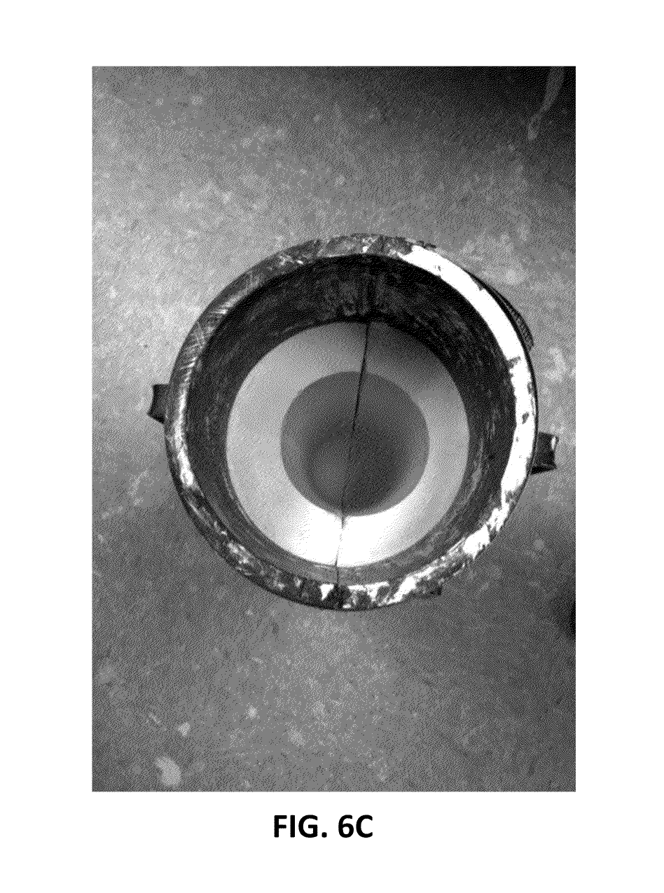

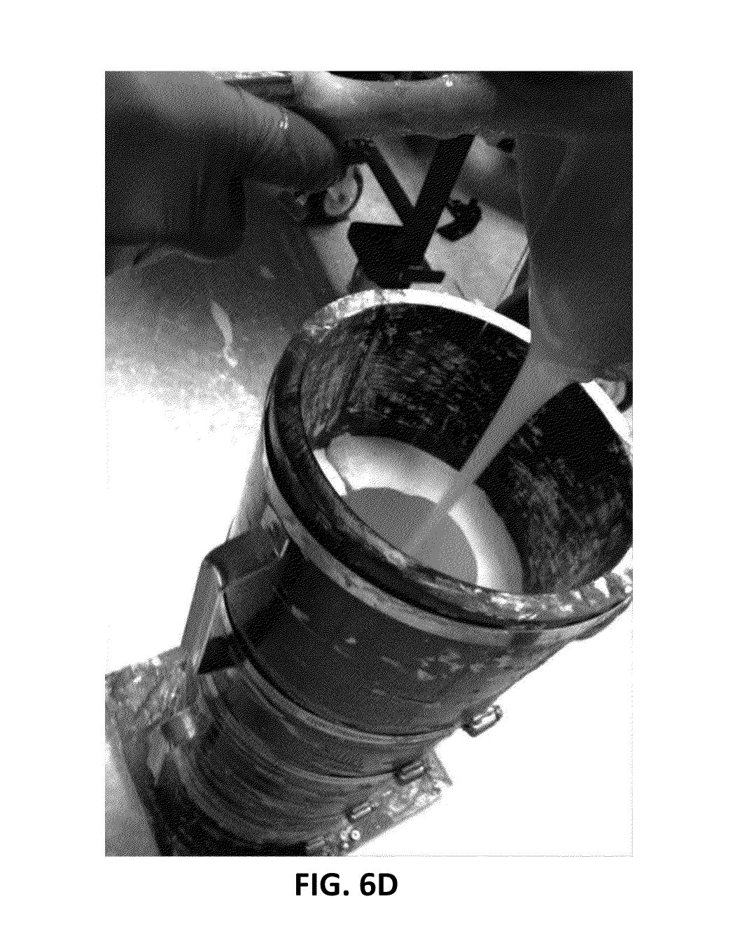

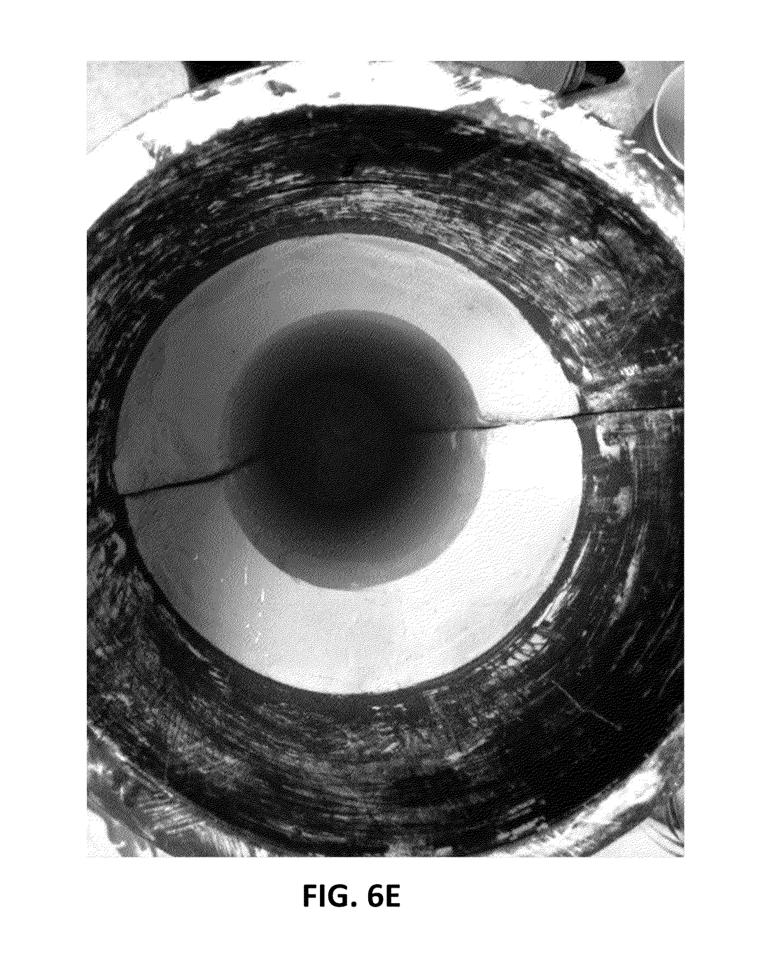

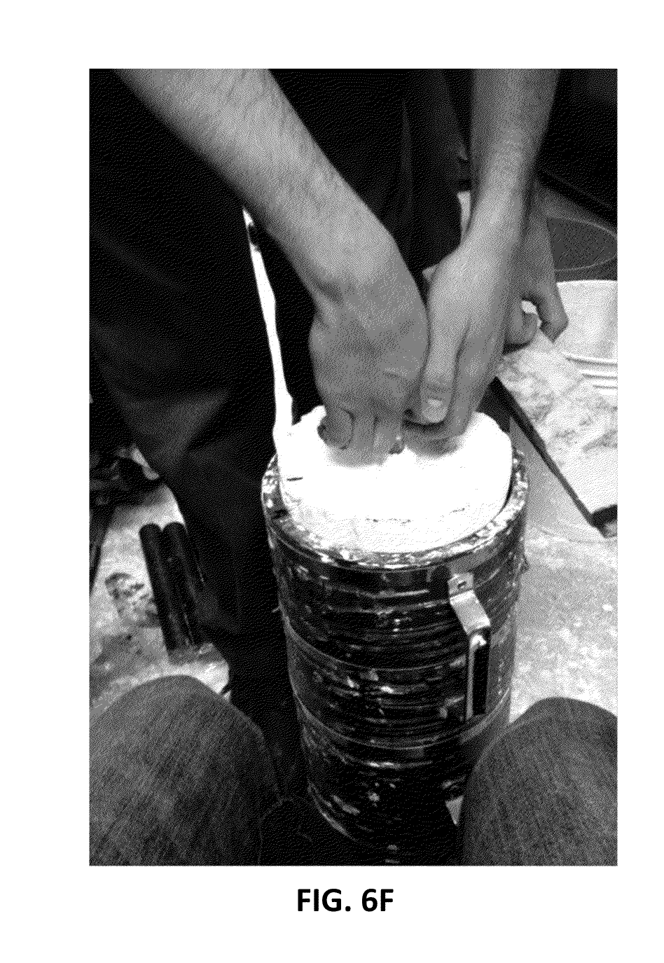

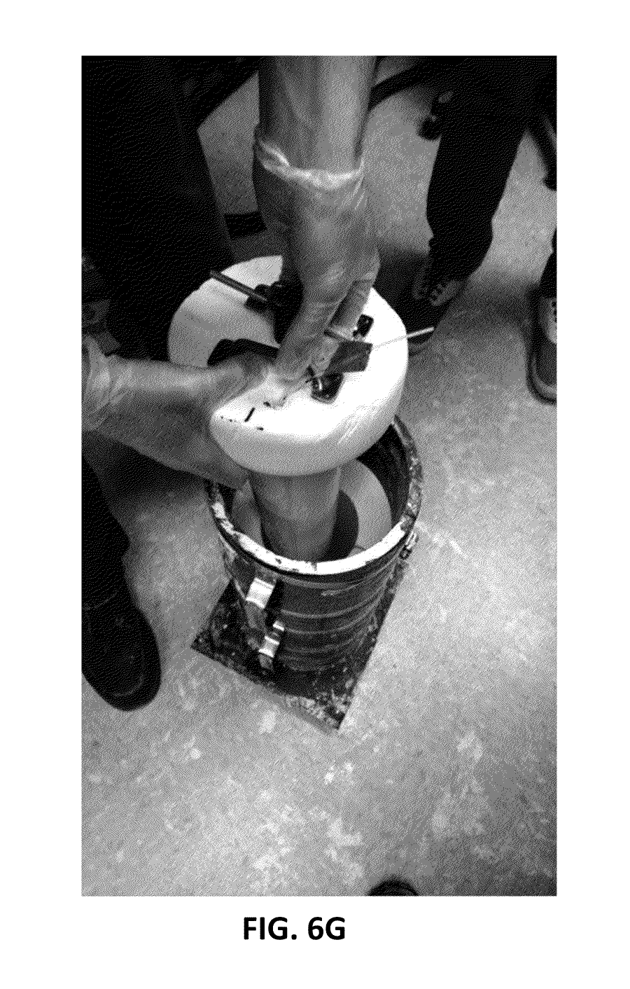

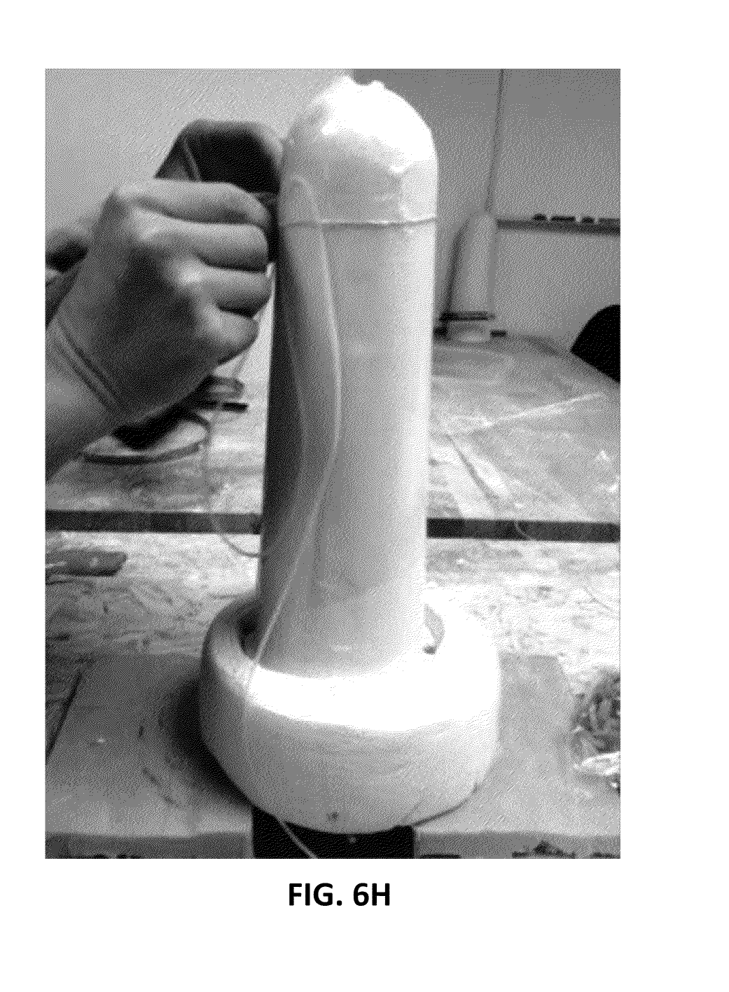

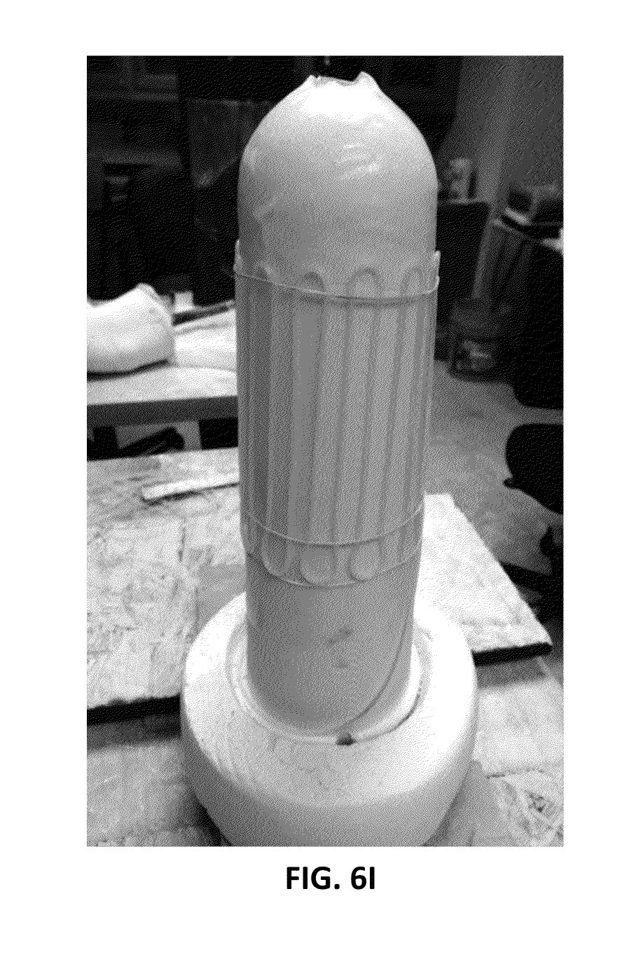

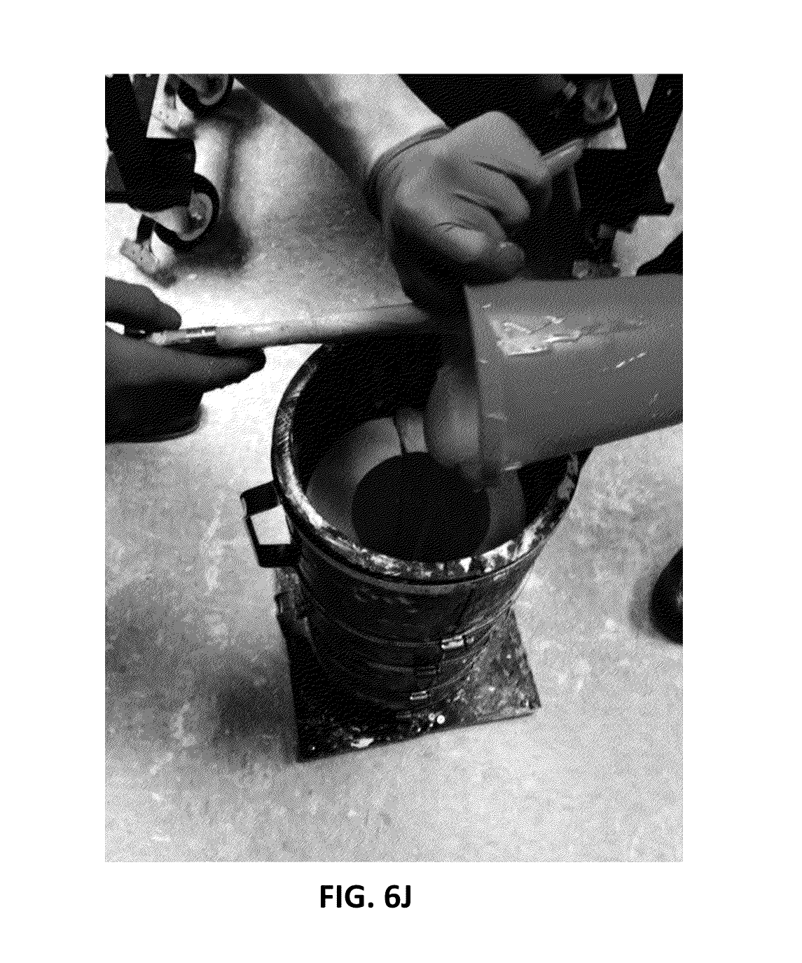

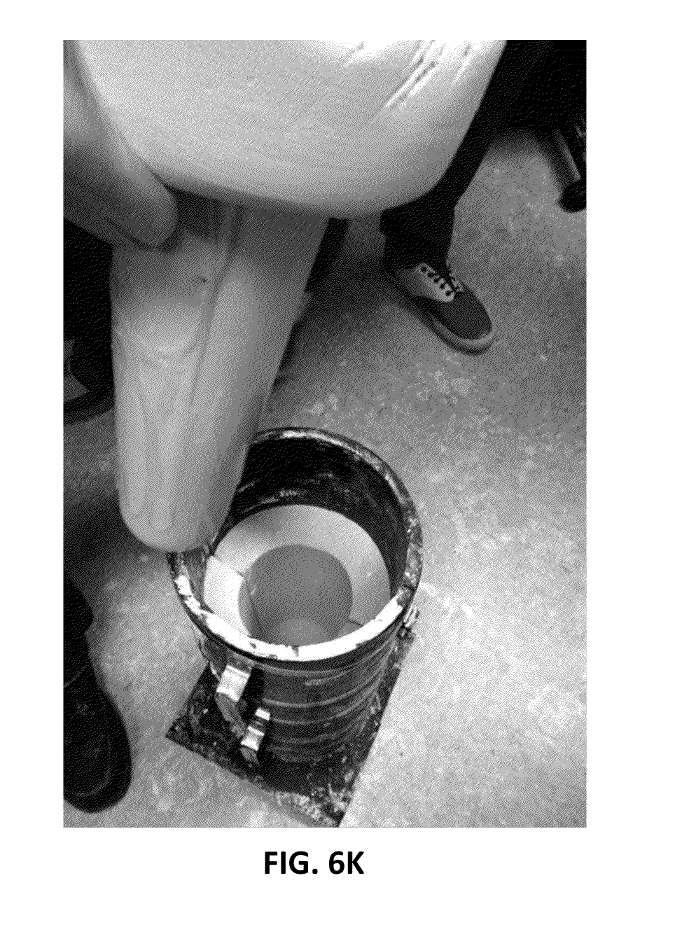

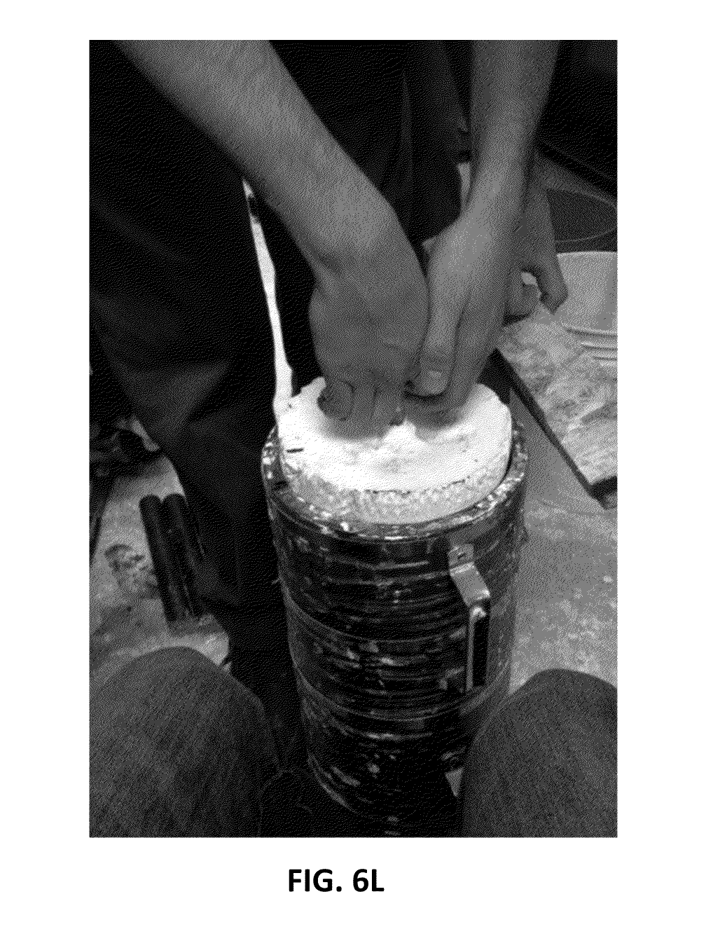

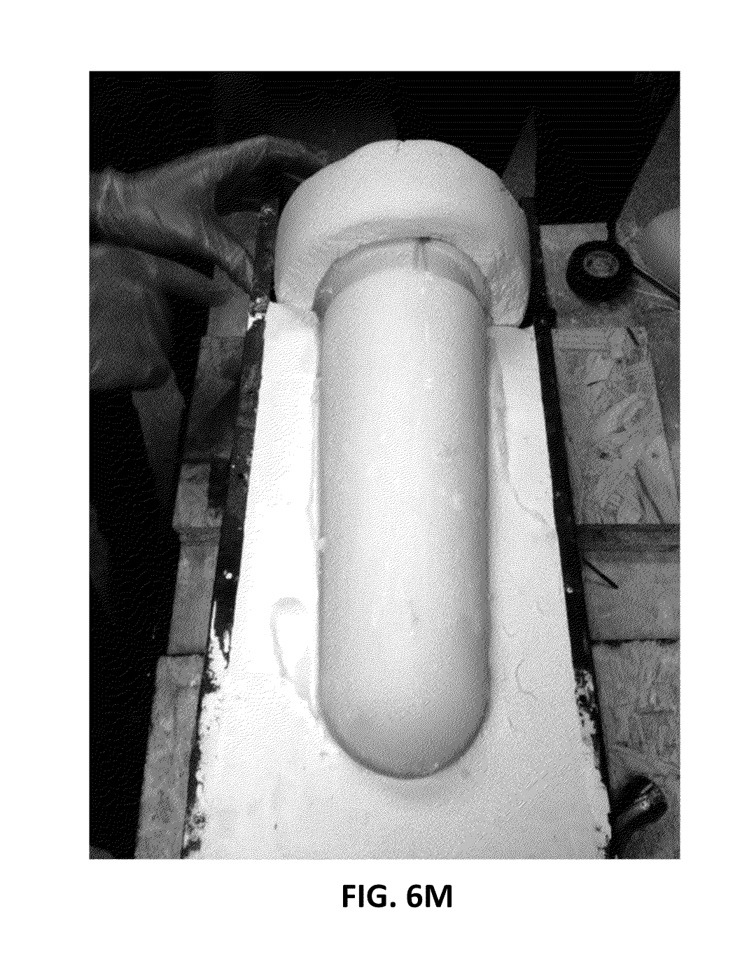

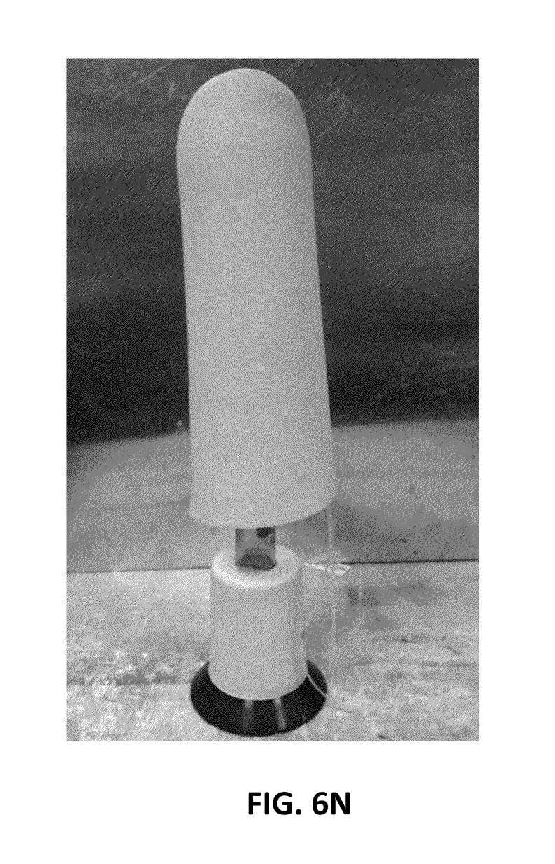

FIGS. 6A-6N illustrate images of steps of manufacturing a prosthetic cooling liner, according to an embodiment of the present invention. FIG. 6A illustrates a model of the user's residual limb. The model can be 3D printed, obtained from a cast, of the residual limb, or any other suitable means of modeling known to or conceivable by one of skill in the art. FIG. 6B illustrates a three part mold for creating the liner, including a core having the same shape as the model of the user's residual limb. FIG. 6C illustrates the two side pieces of the mold ready to receive a layer of silicone in order to form a first layer of the liner, and FIG. 6D illustrates the silicone being poured into the mold in order to form the first layer of the liner. FIG. 6E illustrates the silicone disposed within the mold, and FIG. 6F illustrates the core of the mold being dipped into the side pieces of the mold in order to create the first layer of the liner. FIG. 6G illustrates curing the silicone for the first layer of the liner. FIG. 6H illustrates the fluid conduit being arranged on the first layer of the silicone in the proper alignment for the user. In this example, the fluid conduit is arranged for a tibial prosthesis. The fluid conduit can take the form of tubing embedded in the silicone or positioned on an outside surface of the silicone liner. Alternately, the fluid conduit can be defined by the silicone by a material that can be removed physically or by heat or chemical treatment, leaving behind only a lumen for fluid transfer, defined by the silicone. FIG. 6I illustrates a layer of silicone painted over the fluid conduit. FIG. 6J illustrates the silicone being poured for the outer layer of silicone of the liner. FIGS. 6K and 6L illustrate the core being dipped into the side pieces of the mold in order to form the outer layer of the silicone over the fluid conduit. FIGS. 6M and 6N illustrate the mold pieces being separated and the liner being removed.

Generally, to form the liners, silicone is poured into the bottom of parts one and two. Then the core is dipped into the mold to create a thin layer of silicone on the core. After this layer has cured, the tubes are aligned on the core, fixated with rubber bands, and silicone is painted over the tubes. As the silicone cures, the tubes become held in place, and the rubber bands are removed. The core is then inserted into the mold again with more silicone and allowed to sit for the full cure time of the silicone. This creates the targeted dimensions for the liner, which is removed when the mold is taken apart. The process can be seen in FIGS. 6A through 6N.

In order to use the system, a user must make sure that the liner and the backpack are connected by inspecting the four fittings on the coolant tubes. The user takes the bladder out of the backpack, unscrews the cap and fills the bladder up with ice and then water making sure to fill to the brim of the filling cap. The filling cap is configured to be fluid tight, such that there is no leakage. A gasket or washer can also be used to further enforce the seal. This is necessary so both tubes pull ice water and not air during the process. The user then re-attaches the lid and place bladder in backpack. The user sprays the outside of the liner with alcohol lubricant and then dons the liner by first inverting the liner and then rolling it up the limb trying to remove as much air, keeping tube exit on the outside. Alternately, the liner can be configured to sit atop another liner or on the outside of the prosthesis. The socket and the outer liner are attached and the user makes sure that the tubes are in a comfortable location on the bottom of the outer liner. The system can then be turned on and the system used.

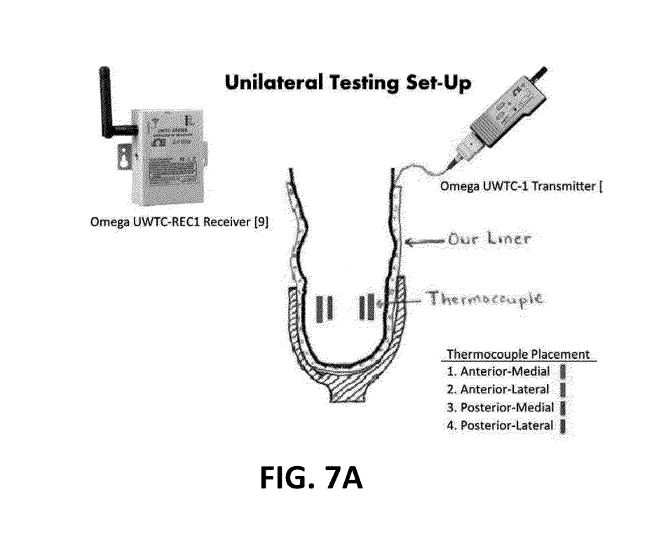

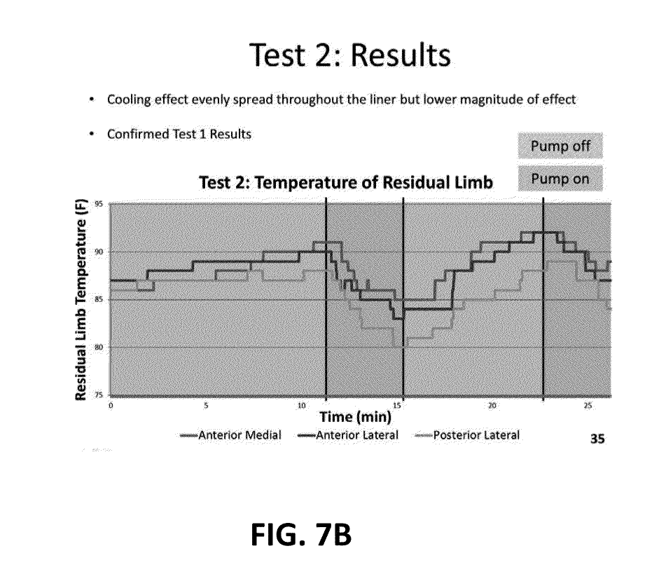

FIGS. 7A and 7B illustrate a sectional view of a setup for an experiment according to an embodiment of the present invention and a graphical view of results of the experiment, respectively. The testing was done with a unilateral amputee. However, any amputee could benefit from the system of the present invention. The amputee wore the system while the cooling feature was cycled on an off. Thermocouples and wireless data loggers were attached to four points on the limb in order to measure skin temperature. The result indicate distribution of the cooling effect. A similar arrangement can be used to implement a thermostat as a component of the present invention.

In another experiment, the user used the system during an hour long strength training workout. Temperature data was not gathered but instead qualitative data was recorded. This simplified the test for the user and simulated the actual usage scenario. The system was easy to use. The user reported that he just added some ice to the pack as he left for the gym. That ice lasted him the entire workout. The liner system was similar to his current set up for putting on except that it required lubrication (not uncommon for liner usage). During the workout he cycled the system on and off and could readily notice the cooling effect. He commented that he would use this system if it was available to him when he would work out in the gym.

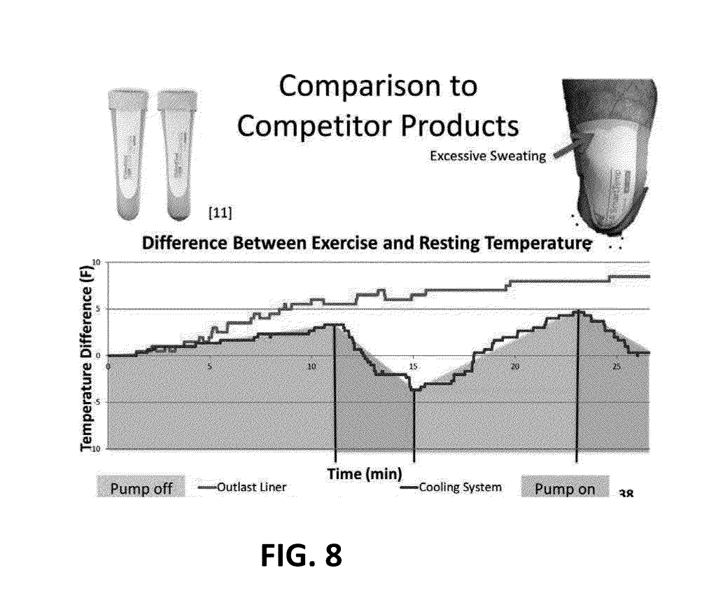

FIG. 8 illustrates a graphical view of a comparison of the present invention with existing products. After testing the cooling liner system with the user, another experiment was performed to test the new Willow Wood Alpha® SmartTemp™ liner. The exact same test was performed with the Willow Wood liner and measured the skin temperature within the liner. The above figure is the comparison of the user's skin temperature with the cooling liner system of the present invention versus the Willow Wood Alpha® SmartTemp™ liner. It is evident from the results that the cooling liner system of the present invention is able to keep the skin temperature of the limb much cooler than the Willow Wood Alpha® SmartTemp™ liner.

The system could also include a temperature feedback system for monitoring temperature of the user's limb to maintain a cool and consistent temperature in the liner. Thermocouples could be embedded in the liner and could provide temperature feedback to an Arduino that controls the coolant fluid flow across the limb. Other improvements could include thermally isolating the bladder and the pump from each other and the user and/or allowing for variations in thickness of the liner.

The many features and advantages of the invention are apparent from the detailed specification, and thus it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly all suitable modifications and equivalents may be resorted to, falling within the scope of the invention.

What is claimed is:

1. A system for cooling comprising:
a liner configured to be disposed between a residual limb of a user and a socket of a prosthetic limb;
a conduit configured for flow of a cooling fluid, wherein the conduit defines an inner lumen, and wherein the inner lumen of the conduit accommodates the flow of the cooling fluid;
a pump in fluid communication with the conduit, such that the pump generates the flow of the cooling fluid through the conduit;
a bladder in fluid communication with the conduit and the pump, wherein the bladder contains a supply of the cooling fluid.

2. The system of claim 1 wherein the liner is formed from one selected from a group consisting of silicone, polyurethane, and a copolymer.

3. The system of claim 1 wherein the conduit takes the form of tubing.

4. The system of claim 3 wherein the tubing is embedded in the liner.

5. The system of claim 3 wherein the tubing is formed from one selected from a group consisting of silicone, polyurethane, and a copolymer.

6. The system of claim 1 wherein the conduit is arranged in a vertical alignment.

7. The system of claim 1 wherein the conduit terminates within a range of approximately 0.25 inches from a distal end of the liner to approximately 4 inches from the distal end of the liner.

8. The system of claim 1 wherein the pump takes the form of a peristaltic pump.

9. The system of claim 1 further comprising a power source configured to provide power to the pump.

10. The system of claim 9 wherein the power source takes the form of a battery.

11. The system of claim 1 wherein the bladder comprises inflow and outflow tubes, and wherein the inflow and outflow tubes are in fluid communication with the pump and the conduit.

12. The system of claim 1 wherein the cooling fluid takes the form of one selected from a group consisting of ice water, water, ice, gas, and liquid containing an additive.

13. The system of claim 1 further comprising a backpack for housing the pump and bladder.

14. The system of claim 10 further comprising a charger for the battery.

15. The system of claim 8 wherein the pump is actuated by one selected from a group consisting of walking, running, and movement.

16. The system of claim 9 further comprising an on/off switch configured to provide control over power delivered to the system.

17. The system of claim 3 wherein the tubing is positioned on an outside surface of the liner.

18. The system of claim 1 further comprising a thermostat.

19. The system of claim 18 wherein the thermostat is configured to cycle the system on and off in order to maintain the temperature of the system at a predetermined temperature.

20. The system of claim 1 further comprising the liner defining the lumen of the conduit.
